(19) United States
(12) Patent Application Publication
(10) Pub. No.: US 2026/0232546 A1
(43) Pub. Date: Aug. 13, 2026

(54) COMPOSITION COMPRISING OIL CAPSULES

(71) Applicant: AMOREPACIFIC CORPORATION, Seoul (KR)

(72) Inventors: Seongchan EO, Yongin-si (KR); Juhee HA, Yongin-si (KR); Seunghan PARK, Yongin-si (KR)

(73) Assignee: AMOREPACIFIC CORPORATION, Seoul (KR)

(21) Appl. No.: 19/292,188

(22) Filed: Aug. 6, 2025

(30) Foreign Application Priority Data
Feb. 12, 2025 (KR) 10-2025-0018236

(51) Int. Cl. A61K 8/11; A61K 8/34; A61K 8/42; A61K 8/63; A61K 8/92; A61Q 19/00

(52) U.S. Cl. CPC A61K 8/11; A61K 8/342; A61K 8/42; A61K 8/63; A61K 8/92; A61Q 19/00; A61K 2800/48

(57) ABSTRACT

A cosmetic composition includes an oil phase having a form of oil capsules; and an aqueous phase, wherein the oil phase includes a lipid component including ceramide or a derivative thereof, cholesterol, and a fatty acid; an alcohol having 16 to 20 carbon atoms; and an oil thickener, and the oil capsules have a transparent appearance.

### CROSS-REFERENCE TO RELATED APPLICATION

This application claims priority to and the benefit of Korean Patent Application No. 10-2025-0018236 filed with the Korean Intellectual Property Office on Feb. 12, 2025, the entire contents of which are incorporated herein by reference.

### BACKGROUND OF THE INVENTION

### (a) Field of the Invention

The present invention relates to a cosmetic composition including oil capsules.

### (b) Description of the Related Art

As the main components of skin lipids, ceramides, cholesterol, and fatty acids have been reported to have various physiological functions, such as forming a skin barrier and protecting the skin from external stimuli, and are materials that are attracting attention in cosmetics.

These skin lipid components are insoluble and, when included in cosmetics, may cause problems such as reduced formulation stability and precipitation. To solve these problems, methods such as using excessive surfactants, using special equipment such as high-pressure emulsification, and encapsulation through a spray drying process have been used.

However, there are problems such as skin irritation when excessive surfactants are used, and the manufacturing method is cumbersome and uneconomical when special equipment is used. In addition, when using a general encapsulation method, there are problems such as a foreign body sensation when applied to the skin, a heavy feel of use when used, or a poor aesthetic appearance of the cosmetic.

### SUMMARY OF THE INVENTION

An embodiment provides a cosmetic composition including oil capsules having a transparent appearance while maintaining a stable formulation.

An embodiment provides a cosmetic composition including an oil phase having a form of oil capsules; and an aqueous phase, wherein the oil phase includes a lipid component including ceramide or a derivative thereof, cholesterol, and a fatty acid; an alcohol having 16 to 20 carbon atoms; and an oil thickener, wherein the oil capsules have a transparent appearance.

An average size of the oil capsules may be from 0.1 mm to 1.5 mm.

The oil capsules may have a spherical shape.

The lipid component may be included in an amount of less than 20 parts by weight based on 100 parts by weight of the alcohol having 16 to 20 carbon atoms.

The alcohol having 16 to 20 carbon atoms may be a branched alcohol.

The alcohol having 16 to 20 carbon atoms may include at least one of hexyldecanol, octyldodecanol, heptyldecanol, octyldecanol, nonyldecanol, butyldodecanol, pentyldodecanol, hexyldodecanol, heptyldodecanol, pentylundecanol, hexylundecanol, heptylundecanol, octylundecanol, and nonylundecanol.

The alcohol having 16 to 20 carbon atoms may be included in an amount of 40 to 70 parts by weight based on 100 parts by weight of the oil phase.

The oil thickener may include at least one of a triglyceride compound, a grafted polyolefin including an aliphatic group of C14 or more, a condensate of castor oil and an alicyclic polyisocyanate, and a condensate of a polyhydric alcohol compound and an alicyclic polyisocyanate.

The oil thickener may include a mixture of the triglyceride-based compound, the grafted polyolefin including the aliphatic group of C14 or more, and the condensate of castor oil and the alicyclic polyisocyanate, or a mixture of the triglyceride-based compound, the grafted polyolefin including the aliphatic group of C14 or more, and the condensate of the polyhydric alcohol-based compound and the alicyclic polyisocyanate.

The oil thickener may have a crosslinked structure.

The oil thickener may have a weight average molecular weight of greater than or equal to 1,000 g/mol.

The oil thickener may be included in an amount of 5 parts by weight to 30 parts by weight based on 100 parts by weight of the oil phase.

The oil phase may be included in an amount of 1 wt % to 9 wt % based on a total weight of the cosmetic composition.

The aqueous phase may include a water-soluble thickener.

The water-soluble thickener may include at least one of an acrylate-based thickener and a carbomer-based thickener.

The water-soluble thickener may be included in an amount of 0.05 wt % to 1.0 wt % based on a total weight of the cosmetic composition.

The cosmetic composition may be an oil in water.

The cosmetic composition may not include a surfactant.

A viscosity of the cosmetic composition may be 500 cps to 10,000 cps.

A cosmetic composition according to an embodiment may stably include a lipid component while providing visual differentiation and aesthetics with a transparent appearance.

In addition, the cosmetic composition according to an embodiment may reduce skin irritation caused by a surfactant and provide excellent feel of use.

### BRIEF DESCRIPTION OF THE DRAWINGS

FIG. 1A is a photograph showing the appearance of Preparation Example 1-1.

FIG. 1B is a photograph showing the appearance of Preparation Example 1-2.

FIG. 1C is a photograph showing the appearance of Preparation Example 1-5.

FIG. 1D is a photograph showing the appearance of Preparation Example 1-6.

FIG. 1E is a photograph showing the appearance of Preparation Example 1-7.

FIG. 1F is a photograph showing the appearance of Preparation Example 1-8.

FIG. 2A is a photograph showing the appearance of Preparation Example 2-1.

FIG. 2B is a photograph showing the appearance of Preparation Example 2-6.

FIG. 3A is a photograph showing the appearance of Example 1.

FIG. 3B is a photograph showing the appearance of Example 2.

FIG. 3C is a photograph showing the appearance of Comparative Example 1.

FIG. 4 is a photograph showing the appearance of Example 1.

### DETAILED DESCRIPTION OF THE EMBODIMENTS

Hereinafter, embodiments of the present disclosure will be described in detail so that a person having ordinary skill in the art to which the present disclosure pertains can easily implement the present disclosure. However, the present disclosure may be implemented in many different forms and is not limited to the embodiments described herein.

The terminology used herein is for the purpose of describing example embodiments only and is not intended to be limiting of the invention. The singular expression includes the plural expression unless the context clearly dictates otherwise.

As used herein, “combination thereof” refers to a mixture, a laminate, a composite, a copolymer, an alloy, a blend, a reaction product, and the like of the constituents.

As used herein, “or” is not to be construed as an exclusive interpretation, for example, “A or B” is construed to include A, B, A+B, and the like.

As used herein, it may be understood that terms such as “comprises,” “includes,” or “have” are intended to designate the presence of an embodied feature, number, step, element, or a combination thereof, but it does not preclude the possibility of the presence or addition of one or more other features, number, step, element, or a combination thereof.

As used herein, “lipid component” refers to a material that has the property of being insoluble in water but soluble in oil, and may provide effects such as improving wrinkles, increasing elasticity, moisturizing, anti-oxidation, whitening, and deodorizing.

As used herein, “oil capsule” refers to a carrier having a structure that, when mixed with water at room temperature and pressure, separates from an aqueous phase including water, includes a lipid component inside, and blocks or inhibits the transfer of substances between the inside and the outside of the capsule. The oil capsules may be expressed as an oil drop or an oil droplet.

As used herein, the term “dispersed” oil capsules is understood that the oil capsules do not coalesce with each other and stably maintain their shape while the oil capsules do not settle to the bottom of the composition but are stably dispersed in the aqueous phase.

As used herein, “transparent” refers to a state in which the shape and color are visible without change when viewed through a medium (composition). Also, the term “transparent” cannot be used interchangeably with the term “translucent,” which refers to a state in which the shape and color of an object are partially changed when viewed through a medium, and the term “opacity,” which refers to a state in which the shape and color of an object are completely invisible when viewed through a medium.

Capsules including lipid components such as ceramide, cholesterol, or fatty acid have the problem of causing a foreign body sensation when applied to the skin, feeling heavy when used, or reducing the aesthetic appearance of the cosmetic. Recently, there have been attempts to develop cosmetics that stably include the lipid components while having excellent feel of use and aesthetics, but there is still no known technology for implementing oil capsules including the lipid components to have high transparency.

Accordingly, the inventors of the present invention have conducted research for several years on a technology that can provide visual differentiation and aesthetics with a transparent appearance while having excellent stability despite including the lipid component, and have finally completed the present invention after experiencing many trials and errors.

Hereinafter, a cosmetic composition according to an embodiment is described.

The cosmetic composition according to an embodiment includes an oil phase having a form of oil capsules; and an aqueous phase, wherein the oil phase includes a lipid component including ceramide or a derivative thereof, cholesterol, and a fatty acid; an alcohol having 16 to 20 carbon atoms; and an oil thickener, and the oil capsules has a transparent appearance.

The lipid component may form a skin barrier and protect the skin from external stimuli. The cosmetic composition according to an embodiment has the characteristics of excellent formulation stability, such as stably including the lipid component in the oil phase having a form of the oil capsules while minimizing the phenomenon of precipitation of the lipid component.

The ceramide or the derivative thereof included in the lipid component may play a role in restoring damaged skin, protecting the skin from external stimuli, and preventing moisture from evaporating from the skin. For example, the derivative of the ceramide may include ceramide NP, ceramide NS, ceramide NH, ceramide AP, ceramide AS, ceramide EOP, ceramide EOS, or a combination thereof, but is not limited thereto.

The ceramide may be included in an amount of 0.1 parts by weight to 10 parts by weight, 0.5 parts by weight to 8 parts by weight, or 0.8 parts by weight to 5 parts by weight based on 100 parts by weight of the oil phase. When the amount of the ceramide satisfies the above range, the effect of ceramide may be secured while the oil capsules may be implemented as a transparent formulation and the formulation stability may be improved.

The cholesterol included in the lipid component may provide flexibility to the lipid layer between skin cells, improve the stability of the skin barrier, and provide a moisturizing effect to the skin. For example, the cholesterol may be included in an amount of 0.5 parts by weight to 13 parts by weight, 0.8 parts by weight to 10 parts by weight, or 1 part by weight to 8 parts by weight based on 100 parts by weight of the oil phase. When the amount of the cholesterol content satisfies the above range, the effect of cholesterol may be secured while improving the formulation stability.

The fatty acid included in the lipid component may play a role in minimizing moisture evaporation from the skin, promoting wound healing and skin regeneration, and maintaining skin elasticity. For example, the fatty acid may include palmitic acid, stearic acid, oleic acid, linoleic acid, alpha-linolenic acid, myristic acid, arachidonic acid, sebacic acid, or a combination thereof, but is not limited thereto.

The fatty acid may be included in an amount of 0.1 parts by weight to 10 parts by weight, 0.5 parts by weight to 8 parts by weight, or 0.8 parts by weight to 5 parts by weight based on 100 parts by weight of the oil phase. When the amount of the ceramide satisfies the above range, the oil capsules may be implemented in a transparent formulation while securing the above effect of fatty acid.

For example, the lipid component may be included in an amount of less than 20 parts by weight, for example, less than or equal to 18 parts by weight, less than or equal to 16 parts by weight, or less than or equal to 15 parts by weight based on 100 parts by weight of the alcohol having 16 to 20 carbon atoms, and may be included in an amount of greater than 10 parts by weight, greater than or equal to 10.5 parts by weight, or greater than or equal to 11 parts by weight based on 100 parts by weight of the alcohol having 16 to 20 carbon atoms. When an amount of the lipid component satisfies the above range, the oil capsules may be implemented as a transparent formulation while ensuring the effects of skin barrier formation and skin protection, and above all, the formulation stability may be greatly improved by suppressing the precipitation of the lipid component.

For example, the lipid component may be included in an amount of 0.5 parts by weight to 20 parts by weight, 1 part by weight to 15 parts by weight, or 3 parts by weight to 12 parts by weight based on 100 parts by weight of the oil phase. When the amount of the lipid component satisfies the above range, the formulation stability may be improved while ensuring the effects of skin barrier formation and skin protection.

The alcohol having 16 to 20 carbon atoms may be capable of enabling the oil capsules to be implemented in a transparent formulation, and improving formulation stability by minimizing precipitation of lipid components, as a solvent. Specifically, when formulating the lipid component into the capsule formulation, it is not easy to improve the formulation stability and at the same time to ensure that the oil capsules has a transparent appearance.

The cosmetic composition according to an embodiment may simultaneously achieve formulation stability and improved transparency of oil capsules by controlling the type of alcohol as a solvent, specifically, the carbon number of the alcohol. For example, if the carbon number of the alcohol is less than 16 or greater than 20, the precipitating phenomenon of lipid component may occur or the transparency of the oil capsules may deteriorate.

The alcohol having 16 to 20 carbon atoms may be a branched alcohol. In this case, the formulation stability and transparency of the oil capsules may be improved. For example, the alcohol having 16 to 20 carbon atoms may include at least one of hexyldecanol, octyldodecanol, heptyldecanol, octyldecanol, nonyldecanol, butyldodecanol, pentyldodecanol, hexyldodecanol, heptyldodecanol, pentylundecanol, hexylundecanol, heptylundecanol, octylundecanol, and nonylundecanol. Specifically, the alcohol having 16 to 20 carbon atoms may include at least one of 2-hexyldecanol, 2-octyldodecanol, 2-heptyldecanol, 2-octyldodecanol, 2-nonyldecanol, 2-butyldodecanol, 2-pentyldodecanol, 2-hexyldodecanol, 2-heptyldodecanol, 2-pentylundecanol, 2-hexylundecanol, 2-heptylundecanol, 2-octylundecanol, and 2-nonylundecanol. More specifically, the alcohol having 16 to 20 carbon atoms may include at least one of hexyldecanol and octyldodecanol.

For example, the alcohol having 16 to 20 carbon atoms may be a mixture of two or more different branched alcohols. When two or more types of branched alcohols having 16 to 20 carbon atoms are used in combination, it may be more advantageous in improving the transparency of the oil capsules than when one type of branched alcohol having 16 to 20 carbon atoms is used alone.

The alcohol having 16 to 20 carbon atoms may be included in an amount of 40 parts by weight to 70 parts by weight, for example, 45 parts by weight to 68 parts by weight, 50 parts by weight to 66 parts by weight, or 55 parts by weight to 65 parts by weight based on 100 parts by weight of the oil phase. When the amount of alcohol having 16 to 20 carbon atoms satisfies the above range, the transparency of the oil capsules may be improved and the formulation stability may be maximized. Specifically, when the alcohol having 16 to 20 carbon atoms is included in an amount of less than 40 parts by weight based on 100 parts by weight of the oil phase, the relative amount of the alcohol having 16 to 20 carbon atoms, which acts as a solvent, is too low, which may cause a problem in that the lipid component is precipitated. In addition, when the alcohol having 16 to 20 carbon atoms is included in an amount of greater than 70 parts by weight based on 100 parts by weight of the oil phase, the viscosity of the oil phase may be significantly reduced (even if a thickener is included), which may impair the stability of the formulation. In addition, the poor stability of the formulation may also negatively affect the transparency of the oil capsule.

The oil thickener may appropriately control the viscosity of the oil phase, thereby minimizing precipitation of the lipid component and improving formulation stability. In addition, the oil thickener can not only implement an oil capsules in a transparent formulation, but also further improve the aesthetics by controlling the particle size of the oil capsule.

A cosmetic composition according to an embodiment may simultaneously achieve formulation stability and improved transparency of oil capsules by selecting the type of the oil thickener. For example, the oil thickener may include at least one of a triglyceride compound, a grafted polyolefin containing an aliphatic group of C14 or more, a condensate of castor oil and an alicyclic polyisocyanate, and a condensate of a polyhydric alcohol compound and an alicyclic polyisocyanate. For example, the oil thickener may include at least one of caprylic/capric triglyceride, hydrogenated poly(C6-20 olefin), HDI/trimethylol hexyllactone crosspolymer, and a castor oil/IPDI copolymer, but is not limited thereto.

For example, the oil thickener may include a mixture of the triglyceride-based compound, the grafted polyolefin including the aliphatic group of C14 or more, and the condensate of castor oil and the alicyclic polyisocyanate, or a mixture of the triglyceride-based compound, the grafted polyolefin including the aliphatic group of C14 or more, and the condensate of the polyhydric alcohol-based compound and the alicyclic polyisocyanate. For example, the oil thickener may include a mixture of caprylic/capric triglyceride, hydrogenated poly(C6-20 olefin), and HDI/trimethylolhexyl lactone crosspolymer, or a mixture of caprylic/capric triglyceride and castor oil/IPDI copolymer, but is not limited thereto.

For example, the oil thickener may have a crosslinked structure. Here, the crosslinked structure may refer to a structure in which a chemical bond is formed between polymer chains to form a three-dimensional network, and the oil thickener used in the cosmetic composition according to an embodiment may have a very high weight average molecular weight, for example, a weight average molecular weight of greater than or equal to 1,000 g/mol, by having the crosslinked structure. For example, the weight average molecular weight of the oil thickener may be less than or equal to 100,000 g/mol, but is not limited thereto. In particular, the oil thickener having the crosslinked structure may have a more effective viscosity control function than an oil thickener not having the crosslinked structure. That is, compounds that do not have a crosslinked structure, such as dextrin palmitate, may not be suitable for use as an oil thickener in a cosmetic composition according to an embodiment.

The oil thickener may be included in an amount of 5 parts by weight to 30 parts by weight, for example, 7 parts by weight to 25 parts by weight, or 10 parts by weight to 20 parts by weight based on 100 parts by weight of the oil phase. When the amount of the oil thickener satisfies the above range, it is effective in stabilizing the lipid component, and the transparency and formulation stability of the oil capsules may be secured at the same time.

For example, the oil phase may further include oil. The oil may include at least one of triglyceride-based oil, hydrocarbon-based oil, ester-based oil, diester-based oil, and silicone-based oil.

The triglyceride oil may include at least one of caprylic/capric triglyceride, caprylic/capric/stearic triglyceride, triethylhexanoin, and tricaprylin.

The hydrocarbon-based oil may include at least one of squalene, hydrogenated polyisobutene, hydrogenated poly(C6-20 olefin), hydrogenated polydecene, paraffin, ceresin, and microcrystalline wax.

The ester-based oil may include at least one of cetyl ethylhexanoate, octyldodecyl myristate, isopropyl palmitate, isopropyl myristate, tocopheryl acetate, butylene glycol dicaprylate/dicaprate, coco-caprylate/caprate, Simmondsia chinensis (Jojoba) seed oil, dicaprylyl carbonate, pentaerythrityl tetraethylhexanoate, and pentaerythrityl tetraisostearate.

The diester-based oil may include at least one of diisostearyl malate, di-C12-13 alkyl malate, neopentyl glycol diheptanoate, tridecyl stearate, neopentyl glycol dicaprylate/dicaprate, tridecyl trimellitate, and dicaprylyl carbonate.

The silicone-based oil may include at least one of dimethicone, cyclomethicone, polydimethylsiloxane (PDMS), methylphenyl polysiloxane, octamethylcyclotetrasiloxane, decamethylcyclopentasiloxane, dodecamethylcyclopentasiloxane, tetradecamethylhexasiloxane, and octamethyltrisiloxane.

The oil phase is in a form of an oil capsule, and the oil capsules have a transparent appearance. For example, transparency may refer to a transmittance of 70% or more, for example, 75% or more, 80% or more, or 85% or more, or 99.9% or less, 99.5% or less, or 99% or less in the visible light region (wavelength 600 nm) in a 1 cm×1 cm quartz cell using a UV-VIS spectrophotometer. The oil capsules are dispersed in the aqueous phase and have a transparent appearance, which can provide visual differentiation and aesthetics.

An average size of the oil capsules may be from 0.1 mm to 1.5 mm, for example from 0.15 mm to 1.45 mm, from 0.2 mm to 1.4 mm, or from 0.3 mm to 1.3 mm. When the average size of the oil capsules satisfies the above range, the oil capsules can provide visual differentiation and aesthetics because the transparency is improved and they can be evenly and stably dispersed in the aqueous phase. Here, the average size of the oil capsules may be measured using photographs taken of the oil capsules using an electron microscope. For example, the average size of oil capsules may be determined by randomly selecting about 20 particles from an electron microscope image, measuring their particle sizes (diameter, major axis, or major axis length), and then taking the average value. The electron microscope may be a transmission electron microscope or a scanning electron microscope.

The oil capsules may have a spherical shape. Here, “spherical shape” may include not only a perfect sphere with a smooth surface, but also a polyhedron that is close to a perfect sphere. Since the oil capsules have a transparent appearance and are implemented in a spherical shape, better visual differentiation and aesthetics may be provided.

For example, the sphericity of the oil capsules may be greater than or equal to 0.7, greater than or equal to 0.75, greater than or equal to 0.8, or greater than or equal to 0.85, and less than or equal to 1.0, less than or equal to 0.98, or less than or equal to 0.95. When the sphericity of the oil capsules satisfies the above range, it is understood that the oil capsules have a shape close to a sphere, and thus visual differentiation and aesthetics may be further improved. Here, the sphericity of the oil capsules may be measured using photographs taken of the oil capsules using an electron microscope. For example, a ratio (r/R) of the particle size (diameter, major axis, or major axis length, r) of the oil capsule to the diameter (R) of a circle having the same area as the projected area of the oil capsule may be taken as the sphericity of the oil capsule.

The oil phase may be included in an amount of 1 wt % to 9 wt %, for example, 1 wt % to 8.5 wt %, or 2 wt % to 8 wt % based on the total weight of the cosmetic composition. When the amount of the oil phase satisfies the above range, the oil capsules may have improved transparency and may be evenly and stably dispersed in the aqueous phase, thereby providing better visual differentiation and aesthetics. Specifically, as the amount (weight) of the oil phase increases, the viscosity of the cosmetic composition including it increases, so that the average size (average particle size) of the oil capsules in the cosmetic composition decreases, which may be advantageous in providing excellent aesthetics. However, if the oil phase is included in an amount of less than 1 wt % based on the total amount of the cosmetic composition, a distribution rate of the oil capsules may be low, thereby deteriorating the aesthetics, and if the oil phase is included in an amount of more than 9 wt % based on the total amount of the cosmetic composition, agglomeration or tangling between the oil capsules (adjacent oil capsules may merge to form larger oil capsules as the liquid film between the oil capsules is destroyed) may occur, or the sphericity or transparency of the oil capsules may be reduced, which may likewise be the main cause of deteriorating the aesthetics.

The aqueous phase may include a water-soluble thickener. The water-soluble thickener may control the viscosity of the aqueous phase to ensure that the oil phase having a form of oil capsules is stably dispersed, and may further improve the aesthetics by controlling the transparency or particle size of the oil capsule.

The water-soluble thickener may include at least one of an acrylate-based thickener and a carbomer-based thickener.

The acrylate-based thickener may include at least one of acrylates/C10-30 alkyl acrylate crosspolymer, ammonium acryloyldimethyltaurate/beheneth-25 methacrylate crosspolymer, acrylates/palmeth-25 acrylate copolymer, acrylates/beheneth-25 methacrylate copolymer, polyacrylate crosspolymer-6, and sodium polyacrylate.

The carbomer-based thickener is a crosslinked acrylic acid polymer, referring to a polymer having an acyclic aliphatic hydrocarbon-based backbone. For example, the carbomer-based thickener may include carbomer, which is commercially available from Lubrizol Corporation under the trade name CARBOPOL®. Examples of carbomers available from Lubrizol Corporation include CARBOPOL 934, 940, 941, 980, 981, 1342, 2984, 5984, SILK 100, ETD 2020, 2050, ULTREZ 10, or ULTREZ 30. However, it is not limited thereto, and for example, the carbomer-based thickener may be one in which an alkaline substance such as triethylamine, potassium hydroxide, or sodium hydroxide is added to cross-link the carbomer.

The water-soluble thickener may further include at least one of a cellulose-based thickener and a polysaccharide-based thickener. The cellulose-based thickener may include at least one of hydroxyethyl cellulose, hydroxypropyl cellulose, hydroxypropyl methylcellulose, methyl cellulose, ethyl cellulose, and carboxymethyl cellulose.

The polysaccharide-based thickener may include at least one of agar, xanthan gum, gellan gum, cellulose gum, tara gum, sclerotium gum, guar gum, carrageenan, and alginate.

The water-soluble thickener may be included in an amount of 0.05 wt % to 1.0 wt % based on the total weight of the cosmetic composition, and may be included in an amount of 0.05 wt % to 0.8 wt %, 0.1 wt % to 0.5 wt %, or 0.2 wt % to 0.4 wt % based on the total weight of the cosmetic composition. When the amount of the water-soluble thickener satisfies the above range, the transparency and particle size of the oil capsules may be adjusted while ensuring formulation stability, thereby further improving the aesthetics. When the amount of the water-soluble thickener is controlled as described above, the viscosity of the cosmetic composition may be appropriately maintained, which may be advantageous in maintaining the average size (particle size) of the oil capsules in the cosmetic composition to 0.1 mm to 1.5 mm.

The cosmetic composition may be an oil in water. Here, “oil in water (O/W)” refers to a formulation in which the continuous phase is an aqueous phase and the dispersed phase is an oil phase in the form of capsules, and is a macroscopically homogeneous formulation. The cosmetic composition may provide excellent visual differentiation and aesthetics because the oil phase having the form of oil capsules is stably dispersed in the aqueous phase without settling to the bottom of the composition while stably maintaining the form of the oil capsule.

The cosmetic composition may not include a surfactant. Here, “does not include” a surfactant is understood that the amount of the surfactant is substantially not included relative to the total cosmetic composition. In addition, the term “substantially does not include” may be understood, but is not limited to, that the amount of the surfactant is less than 1.0 wt %, less than or equal to 0.1%, less than or equal to 0.01%, or less than or equal to 0.001%, or may not be included at all, based on the total weight of the cosmetic composition.

A viscosity of the cosmetic composition may be 500 cps to 10,000 cps, for example 700 cps to 9,000 cps, 1,000 cps to 8,000 cps, or 1,500 cps to 6,000 cps. When the viscosity of the cosmetic composition satisfies the above range, the formulation stability for the oil capsules is secured, while the transparency and particle size of the oil capsules are appropriately controlled, thereby further improving the aesthetics. If the viscosity of the cosmetic composition is less than 500 cps, the oil capsules may not be distributed evenly and may float to the top of the composition, which may result in a deterioration in aesthetics. In addition, if the viscosity of the cosmetic composition exceeds 10,000 cps, the particle size of the oil capsules becomes smaller and the transparency of the oil capsules deteriorates, which may be a factor in lowering the aesthetics. Here, the viscosity of the cosmetic composition may be measured at room temperature (23° C.) using a Brookfield viscometer.

The cosmetic composition may have a transparent appearance. Specifically, since the oil phase having a form of oil capsules has a transparent appearance, a cosmetic composition including the oil phase may also have a transparent appearance, thereby providing visual differentiation and aesthetics. For example, transparency may refer to a transmittance of greater than or equal to 70%, for example, greater than or equal to 75%, greater than or equal to 80%, or greater than or equal to 85%, or less than or equal to 99.9%, less than or equal to 99.5%, or less than or equal to 99% in the visible light region (wavelength 600 nm) in a 1 cm×1 cm quartz cell using a UV-VIS spectrophotometer.

For example, the cosmetic composition may further include additional additives in addition to the aforementioned components as long as it does not impair the purpose of the present invention. For example, the additives may include one or more of a solubilizer, a thickener, a softener, an antioxidant, a stabilizer, a foaming agent, a fragrance, a filler, a metal ion sequestering agent, a chelating agent, a preservative, a vitamin, a skin protectant, a humectant, a dye, and a pigment, as long as the additive is a conventional component.

Hereinafter, examples and comparative examples of the present invention are described. The following examples are merely illustrative of the present invention, and the present invention is not limited to the following examples.

### Preparation Example 1: Preparation of Mixture of Lipid Component and Alcohol (Solvent)

Raw materials listed in Table 1 (unit:weight ratio) were dissolved by stirring and heating at 100° C. to prepare a mixture of lipid components and alcohol (a solvent).

TABLE 1

| | | Prepa- | Prepa- | Prepa- | Prepa- | Prepa- | Prepa- | Prepa- | Prepa- | Prepa- |
|---|---|---|---|---|---|---|---|---|---|---|
| | | ration | ration | ration | ration | ration | ration | ration | ration | ration |
| | | Exam- | Exam- | Exam- | Exam- | Exam- | Exam- | Exam- | Exam- | Exam- |
| | | ple | ple | ple | ple | ple | ple | ple | ple | ple |
| | | 1-1 | 1-2 | 1-3 | 1-4 | 1-5 | 1-6 | 1-7 | 1-8 | 1-9 |
| Lipid | Ceramide NP | 2 | 2 | 2 | 2 | 2 | 2 | 2 | 2 | 2 |
| component | Cholesterol | 4 | 4 | 4 | 4 | 4 | 4 | 4 | 4 | 4 |
| | Palmitic acid* | 2 | 2 | 2 | 2 | 2 | 2 | 2 | 2 | 2 |
| | stearic acid | | | | | | | | | |
| Alcohol | Hexyl decanol | 60 | 30 | 70 | — | — | — | — | 40 | 75 |
| (solvent) | Octyl | — | 30 | — | 60 | — | — | — | — | — |
| | dodecanol | | | | | | | | | |
| | Behenyl | — | — | — | — | 60 | — | — | — | — |
| | alcohol | | | | | | | | | |
| | Caprylic/capric | — | — | — | — | — | 60 | — | — | — |
| | triglyceride | | | | | | | | | |
| | C12-15 | — | — | — | — | — | — | 60 | — | — |
| | alkylbenzoate | | | | | | | | | |

palmitic acid*stearic acid: mixture of palmitic acid and stearic acid

### Preparation Example 2: Preparation of Oil Phase

Raw materials listed in Table 2 (unit:wt %) was dissolved by stirring and heating at 100° C. to prepare each oil phase.

TABLE 2

| | | Prepa- | Prepa- | Prepa- | Prepa- | Prepa- | Prepa- | Prepa- | Prepa- | Prepa- | Prepa- |
|---|---|---|---|---|---|---|---|---|---|---|---|
| | | ration | ration | ration | ration | ration | ration | ration | ration | ration | ration |
| | | Exam- | Exam- | Exam- | Exam- | Exam- | Exam- | Exam- | Exam- | Exam- | Exam- |
| | | ple | ple | ple | ple | ple | ple | ple | ple | ple | ple |
| | | 2-1 | 2-2 | 2-3 | 2-4 | 2-5 | 2-6 | 2-7 | 2-8 | 2-9 | 2-10 |
| Lipid | Ceramide NP | 2 | 2 | 2 | 2 | 2 | 2 | 2 | 2 | 2 | 2 |
| component | Cholesterol | 4 | 4 | 4 | 4 | 4 | 4 | 4 | 4 | 4 | 4 |
| | Palmitic acid*stearic | 2 | 2 | 2 | 2 | 2 | 2 | 2 | 2 | 2 | 2 |
| | acid | | | | | | | | | | |
| Alcohol | Hexyldecanol | 30 | 30 | 30 | 30 | 75 | 30 | 30 | 28 | 30 | 30 |
| (solvent) | Octyldodecanol | 30 | 30 | 30 | 30 | — | 30 | 30 | 28 | 30 | 30 |
| Oil | Caprylic/capric | 17 | 17 | 27 | 12 | 2 | 17 | 31 | 1 | 17 | 17 |
| | triglyceride | | | | | | | | | | |
| Oil | Caprylic/capric | 15 | — | 5 | 20 | 15 | — | 1 | 35 | 7.5 | — |
| thickener | triglyceride*hydrogenated | | | | | | | | | | |
| | poly | | | | | | | | | | |
| | (C6-20 olefin)* | | | | | | | | | | |
| | HDI/trimethylol | | | | | | | | | | |
| | hexyllactone | | | | | | | | | | |
| | crosspolymer | | | | | | | | | | |
| | Caster oil/IPDI | — | 15 | — | — | — | — | — | — | — | 7.5 |
| | copolymer*caprylic/ | | | | | | | | | | |
| | capric triglyceride | | | | | | | | | | |
| | Dextrin palmitate | — | — | — | — | — | 15 | — | — | 7.5 | 7.5 |

| | | | | | | | | | | |
|---|---|---|---|---|---|---|---|---|---|---|
| Total | 100 | 100 | 100 | 100 | 100 | 100 | 100 | 100 | 100 | 100 |

palmitic acid*stearic acid: a mixture of palmitic acid and stearic acid
caprylic/capric triglyceride*hydrogenated poly (C6-20 olefin)* HDI/trimethylol hexyllactone crosspolymer: a mixture of caprylic/capric triglyceride, hydrogenated poly(C6-20olefin), and HDI/trimethylol hexyllactone crosspolymer
casteroil/IPDIcopolymer*caprylic/capric triglyceride: a mixture of casteroil/IPDIcopolymer, and caprylic/capric triglyceride

### Preparation Example 3: Preparation of Cosmetic Composition

Raw materials of an aqueous phase listed in Table 3 (unit:wt %) were dissolved by heating at 70° C. to 80° C., while stirring, and the oil phase was added to the aqueous phase, while still stirring, to manufacture oil capsules. Subsequently, the oil capsules were cooled to 40° C., and a pH controlling agent was added thereto to manufacture cosmetic compositions.

TABLE 3

| | | Example | Example | Example | Example | Comparative | Reference | Reference | Reference | Reference |
|---|---|---|---|---|---|---|---|---|---|---|
| | | 1 | 2 | 3 | 4 | Example 1 | Example 1 | Example 2 | Example 3 | Example 4 |
| Aqueous | Purified | TO 100 | TO 100 | TO 100 | TO 100 | TO 100 | TO 100 | TO 100 | TO 100 | TO 100 |
| phase | water | | | | | | | | | |
| | Chelating | q.s. | q.s. | q.s. | q.s. | q.s. | q.s. | q.s. | q.s. | q.s. |
| | agent | | | | | | | | | |
| | Polyol | 10 | 10 | 10 | 10 | 10 | 10 | 10 | 10 | 10 |
| | Acrylate/ | 0.2 | 0.2 | 0.1 | 0.2 | 0.2 | 0.2 | 0.2 | 0.03 | 2.0 |
| | C10-30 | | | | | | | | | |
| | alkylac- | | | | | | | | | |
| | rylatecrosspoly- | | | | | | | | | |
| | mer | | | | | | | | | |
| | Carbomer | — | 0.1 | 0.25 | — | — | — | — | — | — |
| | Antiseptic | q.s. | q.s. | q.s. | q.s. | q.s. | q.s. | q.s. | q.s. | q.s. |
| Oil | Preparation | 5 | 5 | 5 | 1 | — | 0.5 | 10 | 5 | 5 |
| phase | Example 2-1 | | | | | | | | | |
| | Preparation | — | — | — | — | 5 | — | — | — | — |
| | Example 2-6 | | | | | | | | | |
| pH | Tromethamine | q.s. | q.s. | q.s. | q.s. | q.s. | q.s. | q.s. | q.s. | q.s. |
| controlling | | | | | | | | | | |
| agent | | | | | | | | | | |

| | | | | | | | | | |
|---|---|---|---|---|---|---|---|---|---|
| Total | 100 | 100 | 100 | 100 | 100 | 100 | 100 | 100 | 100 |

### Experimental Example 1: Evaluation of Formulation Stability and Transparency for Preparation Example 1

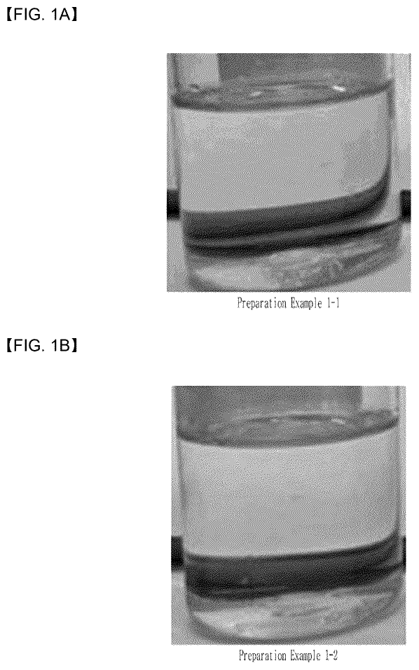

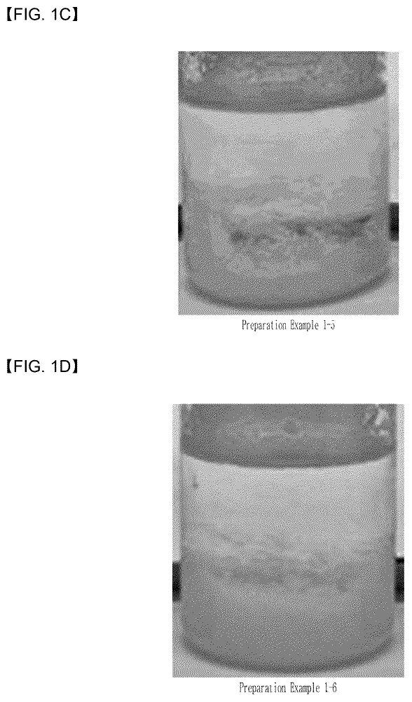

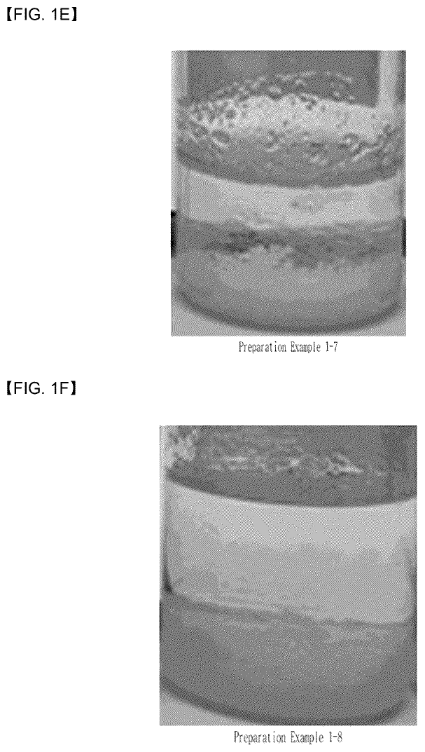

Preparation Example 1 was evaluated with respect to formulation stability and transparency, and the results are shown in Table 4, FIGS. 1A, 1B, 1C, 1D, 1E, and 1F.

### (1) Evaluation of Formulation Stability

Each sample was visually examined one month after the manufacture to determine whether or not the lipid components were precipitated with naked eyes, an image of the sample one month later is shown in FIGS. 1A, 1B, 1C, 1D, 1E, and 1F.

### (2) Transparency of Oil Capsules

The oil capsules dispersed in oil phase were visually inspected and then, evaluated with respect to transparency in the following method.
◯: when the oil capsules showed no shape or color changes
Δ: when the oil capsules had a very small size or were coagulated one another and thus appeared to be suspended
x: when the oil capsules showed shape or color changes due to precipitation of the lipid components

TABLE 4

| | | Lipid | |
|---|---|---|---|
| | | component | |
| | | is precipitated | |
| | | or not | Transparency |
| Mixture of | Preparation Example 1-1 | X | ◯ |
| lipid | Preparation Example 1-2 | X | ◯ |
| component | Preparation Example 1-3 | X | ◯ |
| and alcohol | Preparation Example 1-4 | X | ◯ |
| (solvent) | Preparation Example 1-5 | ◯ | X |
| | Preparation Example 1-6 | ◯ | X |
| | Preparation Example 1-7 | ◯ | X |
| | Preparation Example 1-8 | ◯ | X |
| | Preparation Example 1-9 | X | Δ |

Referring to Table 4, FIGS. 1A, 1B, 1C, 1D, 1E, and 1F, Preparation Examples 1-1 to 1-4 maintained stable formulation without precipitation of the lipid components even after one month, wherein the oil capsules appeared transparent.

On the other hand, Preparation Example 1-5 using alcohol with more than carbon atoms as a solvent and Preparation Examples 1-6 and 1-7 including a generally-used solvent instead of the alcohol exhibited deteriorated formulation stability such as precipitation of the lipid components and the like, wherein the oil capsules appeared opaque.

In addition, Preparation Example 1-8, where the amount of the lipid components to that of the solvent was adjusted to be very high, exhibited deteriorated formulation stability such as precipitation of the lipid components and the like, wherein the oil capsuled appeared opaque.

And, in Preparation Example 1-9, where the amount of the lipid components to that of the solvent was adjusted to be very small, the lipid components were not precipitated, but the oil capsules appeared to be suspended. In addition, as described below, even when Preparation Example 1-9 was mixed by stirring with an oil thickener to manufacture an oil phase (Preparation Example 2-5), because thickening ability of the oil thickener was weakened, the oil capsules had a reduced particle size and became less transparent.

### Experimental Example 2: Evaluation of Formulation Stability and Transparency for Preparation Example 2

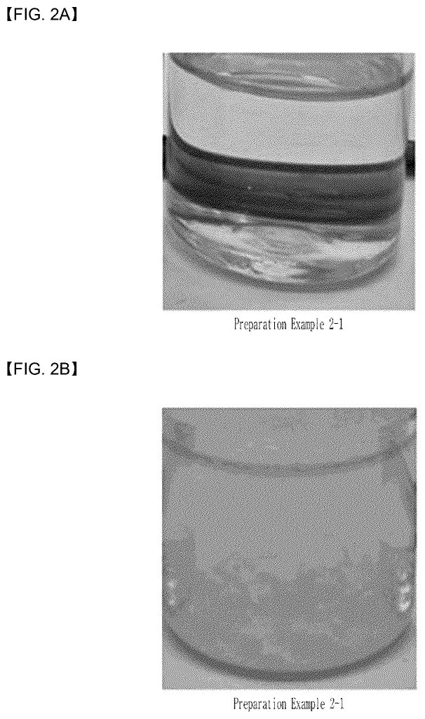

Preparation Example 2 was evaluated with respect to formulation stability and transparency, and the results are shown in Table 5, FIGS. 2A, and 2B. The specific evaluation method was the same as Experimental Example 1.

TABLE 5

| | | Lipid | |
|---|---|---|---|
| | | component | |
| | | is precipitated | |
| | | or not | Transparency |
| Oil | Preparation Example 2-1 | X | ◯ |
| phase | Preparation Example 2-2 | X | ◯ |
| | Preparation Example 2-3 | X | ◯ |
| | Preparation Example 2-4 | X | ◯ |
| | Preparation Example 2-5 | X | Δ |
| | Preparation Example 2-6 | ◯ | X |
| | Preparation Example 2-7 | X | Δ |
| | Preparation Example 2-8 | X | Δ |
| | Preparation Example 2-9 | ◯ | X |
| | Preparation Example 2-10 | ◯ | X |

Referring to Table 5, FIGS. 2A, and 2B, Preparation Examples 2-1 to 2-4 maintained stable formulations without precipitation of the lipid components even after one month, wherein the oil capsules appeared transparent.

On the other hand, Preparation Example 2-5, which was manufactured as an oil phase by stirring Preparation Example 1-9, where the amount of the lipid components was adjusted to be very small to that of the solvent, with an oil thickener together, was confirmed that as thickening ability of the oil thickener was reduced, the oil capsules had a very small size and became less transparent.

Preparation Examples 2-6, 2-9, and 2-10 using dextrin palmitate as an oil thickener were confirmed that formulation stability and transparency were deteriorated due to precipitation of the lipid components and the like. In other words, the transparency was seen to be deteriorated depending on compatibility with the oil thickener with a crosslinked structure, which confirmed that a type of the oil thickener was also a very important factor in terms of formulation stability and transparency.

In addition, in Preparation Example 2-7, where the amount of the oil thickener was adjusted to be very low, the oil capsules were formed to be very small and appeared to be suspended. Furthermore, even in Preparation Example 2-8, where the amount of the oil thickener was adjusted to be very high, the oil capsules were not only formed to have irregular shapes and non-uniform sizes but also coagulated one another and appeared to be suspended, which reduced aesthetic appeal.

### Experimental Example 3: Evaluation of Formulation Stability and Transparency for Preparation Example 3

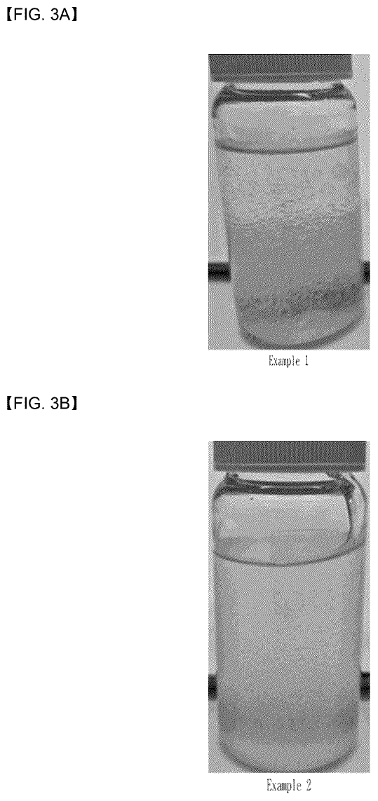

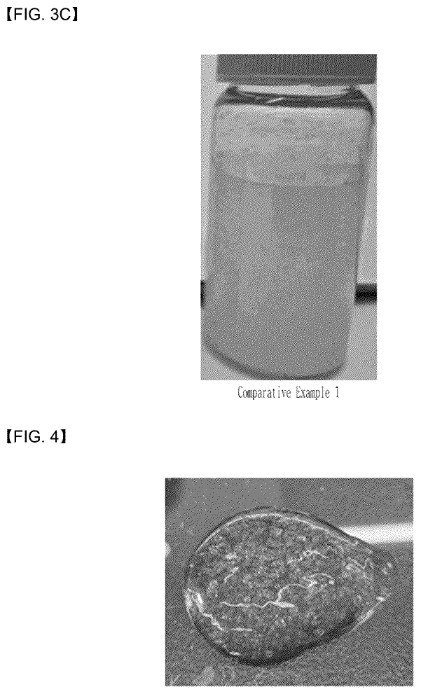

Preparation Example 3 was evaluated with respect to formulation stability and transparency, and the results are shown in Table 6, FIGS. 3A, 3B, and 3C. The specific evaluation method was the same as Experimental Example 1.

TABLE 6

| | | Lipid | |
|---|---|---|---|
| | | component | |
| | | is precipitated | |
| | | or not | Transparency |
| Cosmetic | Example 1 | X | ◯ |
| composition | Example 2 | X | ◯ |
| | Example 3 | X | ◯ |
| | Example 4 | X | ◯ |
| | Comparative Example 1 | ◯ | X |
| | Reference Example 1 | ◯ | — |
| | Reference Example 2 | ◯ | X |
| | Reference Example 3 | ◯ | — |
| | Reference Example 4 | ◯ | X |

Referring to Table 6, FIGS. 3A, 3B, and 3C, Examples 1 to 4, wherein the oil capsules maintained stable formulation without precipitation of the lipid components even after one month and appeared transparent, were confirmed to provide a cosmetic composition with excellent transparency.

On the other hand, in Comparative Example 1 including Preparation Example 2-6 (using dextrin palmitate as an oil thickener), as the oil capsules had less formulation stability such as precipitation of the lipid components and appeared to be suspended, the cosmetic composition also appeared opaque, which reduced aesthetic appeal.

In addition, Reference Example 1, where the amount of the oil phase was adjusted to be low, exhibited deteriorated formulation stability, and transparency of the oil capsules was difficult to check due to the very low amount of the oil capsules in the cosmetic composition. In addition, Reference Example 2, where the amount of the oil phase was adjusted to be high, exhibited insufficient formulation stability, and as the oil capsules were coagulated one another, transparency of the oil capsules and the cosmetic composition also was insufficient.

Reference Example 3, where the amount of acrylate/C10-30 alkyl acrylate crosspolymer as a water-soluble thickener was adjusted to be low, was confirmed to exhibit insufficient formulation stability, and because the aqueous phase had very low viscosity, the oil capsules were floated on the top of the cosmetic composition, making it difficult to check transparency of the oil capsules. In addition, Reference Example 4, where the amount of a water-soluble thickener was adjusted to be high, exhibited deteriorated formulation stability such as precipitation of lipid components and the like, and because the aqueous phase had very high viscosity, the oil capsules were formed very fine, which deteriorated transparency of the cosmetic composition.

### Experimental Example 4: Measurement of Viscosity of Cosmetic Composition and Average Size of Oil Capsules

The cosmetic composition of Preparation Example 3 was measured with respect to viscosity, transparency of oil capsules, and an average size of the oil capsules, and the results are shown in Table 7. The specific measurement method is as follows.

### (1) Viscosity of Cosmetic Composition

The composition was placed at room temperature (23° C.) and then, measured with respect to viscosity at 12 rpm for 2 minutes by using a Brookfield RVDV-III viscometer spindle #3.

(2) Particle size of Oil Capsules

After taking an electron microscope image of the oil capsules, the image was used to select about 20 particles to measure a major length thereof and then, calculate an average value of the measurements.

TABLE 7

| | Viscosity | Particle size of oil capsules |
|---|---|---|
| | (cps) | (mm) |
| Example 1 | 2,200 | 0.8 |
| Example 2 | 4,500 | 0.6 |
| Example 3 | 6,000 | 0.4 |
| Example 4 | 2,000 | 0.5 |
| Comparative | 2,100 | Difficult to measure due to irregular |
| Example 1 | | and uneven particle size |
| Reference | 2,000 | — |
| Example 1 | | |
| Reference | 2,300 | Difficult to measure |
| Example 2 | | due to large deviation in particle size |
| Reference | — | The oil capsules are clumped |
| Example 3 | | together and difficult to measure |
| | | a particle size |
| Reference | 48,000 | 0.05 |
| Example 4 | | |

Referring to Table 7, Examples 1 to 4 exhibited viscosity of 2,000 cps to 6,000 cps, and the oil capsules had a particle size of 0.4 mm to 0.8 mm.

As a representative example, an image showing the appearance of Example 1 was shown in FIG. 4. Referring to FIG. 4, in Example 1, because the oil capsules of appeared transparent, the cosmetic composition had excellent transparency, and also, because the oil capsules with a uniform size, the composition was confirmed to have visual differentiation and aesthetic appeal.

On the other hand, in Reference Example 1, where the amount of the oil capsules was very low in the cosmetic composition, it was difficult to measure a particle size of the oil capsules. In addition, in Reference Example 2, where the oil capsules particle had very severe size deviation, it was difficult to measure an average particle size thereof.

In Reference Example 3, viscosity of the cosmetic composition was impossible to measure due to very low viscosity of the aqueous phase, and because the oil capsules were coagulated one another, it was difficult to measure a particle size of the oil capsules. In addition, in Reference Example 4, because the cosmetic composition had very high viscosity, the oil capsules were formed to have a very small particle size.

### Experimental Example 5: Sensory Evaluation

The cosmetic composition of Preparation Example 3 was evaluated with respect to feel of use and aesthetic appearance, and the results are shown in Table 6. The specific measurement method is as follows.

The cosmetic composition was provided to ten panelists to use for one week. After using it for one week, the panelists were asked to evaluate the cosmetic composition with respect to the feel of use and aesthetic appearance by using a 7-point scale. The feel of use was evaluated by giving the higher score to the lighter feel of use without foreign body sensation. In addition, the aesthetics was evaluated by giving the higher point to the better visual difference and aesthetic appeal in appearance differentiated from those of general cosmetics.

TABLE 8

| | Feel of use | Aesthetic appearance |
|---|---|---|
| Example 1 | 6.1 | 6.4 |
| Example 2 | 5.8 | 6.1 |
| Example 3 | 5.4 | 5.8 |
| Example 4 | 6.1 | 4.9 |
| Comparative Example 1 | 3.1 | 1.9 |
| Reference Example 1 | 3.3 | 3.4 |
| Reference Example 2 | 1.5 | 2.1 |
| Reference Example 3 | 1.9 | 1.1 |
| Reference Example 4 | 2.3 | 1.5 |

Referring to Table 8, Examples 1 to 4 were evaluated to have every excellent feel of use and aesthetic appearance. In particular, referring to FIGS. 3A, 3B, 3C, and 4, the oil capsules of the examples had transparent appearance and thus were confirmed to provide visual differentiation and aesthetics.

On the other hand, Comparative Example 1 and Reference Examples 1 to 4 were evaluated to have inferior feel of use and aesthetic appearance. In particular, referring to FIGS. 3A, 3B, and 3C, because the oil capsules of the comparative example had opaque appearance, aesthetics of the composition also were evaluated to be unsatisfactory.

While this invention has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

What is claimed is:

1. A cosmetic composition, including
an oil phase having a form of oil capsules; and an aqueous phase,
wherein the oil phase includes
a lipid component including ceramide or a derivative thereof, cholesterol, and fatty acids;
an alcohol having 16 to 20 carbon atoms; and
an oil thickener, and
the oil capsules have a transparent appearance.

2. The cosmetic composition of claim 1, wherein
an average size of the oil capsules is from 0.1 mm to 1.5 mm.

3. The cosmetic composition of claim 1, wherein
the oil capsules have a spherical shape.

4. The cosmetic composition of claim 1, wherein
the lipid component is included in an amount of less than 20 parts by weight based on 100 parts by weight of the alcohol having 16 to 20 carbon atoms.

5. The cosmetic composition of claim 1, wherein
the alcohol having 16 to 20 carbon atoms is a branched alcohol.

6. The cosmetic composition of claim 5, wherein
the alcohol having 16 to 20 carbon atoms includes at least one of hexyldecanol, octyldodecanol, heptyldecanol, octyldecanol, nonyldecanol, pentyldodecanol, hexyldodecanol, heptyldodecanol, butyldodecanol, pentylundecanol, hexylundecanol, heptylundecanol, octylundecanol, and nonylundecanol.

7. The cosmetic composition of claim 1, wherein
the alcohol having 16 to 20 carbon atoms is included in an amount of 40 to 70 parts by weight based on 100 parts by weight of the oil phase.

8. The cosmetic composition of claim 1, wherein
the oil thickener includes at least one of a triglyceride-based compound, a grafted polyolefin including an aliphatic group of C14 or more, a condensate of castor oil and an alicyclic polyisocyanate, and a condensate of a polyhydric alcohol compound and an alicyclic polyisocyanate.

9. The cosmetic composition of claim 8, wherein
the oil thickener includes a mixture of the triglyceride-based compound, the grafted polyolefin including the aliphatic group of C14 or more, and the condensate of castor oil and the alicyclic polyisocyanate, or a mixture of the triglyceride-based compound, the grafted polyolefin including the aliphatic group of C14 or more, and the condensate of the polyhydric alcohol-based compound and the alicyclic polyisocyanate.

10. The cosmetic composition of claim 1, wherein
the oil thickener has a crosslinked structure.

11. The cosmetic composition of claim 1, wherein
the oil thickener has a weight average molecular weight of greater than or equal to 1,000 g/mol.

12. The cosmetic composition of claim 1, wherein
the oil thickener is included in an amount of 5 parts by weight to 30 parts by weight based on 100 parts by weight of the oil phase.

13. The cosmetic composition of claim 1, wherein
the oil phase is included in an amount of 1 wt % to 9 wt % based on a total weight of the cosmetic composition.

14. The cosmetic composition of claim 1, wherein
the aqueous phase includes a water-soluble thickener.

15. The cosmetic composition of claim 14, wherein
the water-soluble thickener includes at least one of an acrylate-based thickener and a carbomer-based thickener.

16. The cosmetic composition of claim 14, wherein
the water-soluble thickener is included in an amount of 0.05 wt % to 1.0 wt % based on a total weight of the cosmetic composition.

17. The cosmetic composition of claim 1, wherein
the cosmetic composition is an oil in water.

18. The cosmetic composition of claim 1, wherein
the cosmetic composition does not include a surfactant.

19. The cosmetic composition of claim 1, wherein
a viscosity of the cosmetic composition is 500 cps to 10,000 cps.
